(19) United States
(12) Patent Application Publication
(10) Pub. No.: US 2006/0070630 A1
(43) Pub. Date: Apr. 6, 2006

(54) Bite-positioner for sportsmen

(76) Inventor: Rosalba Giannoni, Perugia (IT)

Correspondence Address: Dykema Gossett PLLC;Suite 300 West, 1300 I Street, N.W., Washington, DC, 20005-3306, US

(21) Appl. No.: 11/283,786

(22) Filed: Nov. 22, 2005

Related U.S. Application Data
(63) Continuation-in-part of application No. PCT/IT03/00847, filed on Dec. 19, 2003.

(30) Foreign Application Priority Data
May 26, 2003 (IT) RM2003A000257

(51) Int. Cl. A61F 5/37; A61F 11/00; A61C 5/14; A61F 13/00

(52) U.S. Cl. 128/861; 128/846; 128/857; 128/859; 128/862

(57) ABSTRACT

A bite-positioner comprises a support body defined by two half-parts developing in opposite directions from a median plane and having a first active surface able to be engaged to the lower arch of a user's mouth and a second active surface able to be engaged to the upper arch of the user's mouth. The first active surface is orthogonal to said median plane, substantially U-shaped and defined by two half-surfaces, each associated to a respective half-part of the support body, the second active surface is orthogonal to said median plane, substantially U-shaped and defined by two half-surfaces, each associated to a respective half-part of the support body. The active surfaces develop substantially parallel to each other and mutually separated by a determined distance defining a useful thickness of said bite-positioner. The useful thickness of at least a bearing portion of one of the half-parts is greater than the useful thickness of the other of the two half-parts.

### TECHNICAL FIELD

The present invention relates to a bite-positioner for golf players, tennis players, as well as athletes playing sports that require muscular stress of legs or arms and that are reflected also in temporal-mandibular joints.

The term “bite-positioner” means a device which can be inserted between the lower and upper occlusal plane of a user's mouth, able to maintain the jaw in a determined position when the teeth are tightly shut on the device itself.

In the field of sports, the bite-positioner is the means whereby the athletes obtains an optimal dental bearing which has favourable repercussions on the efficiency of the athletic gesture and hence on the results which the athlete is able to obtain.

In recent years, the observation of frequent associations between dental-maxillo-facial alterations and dysfunctions of the spine and of postural balance and of interactions between the biomechanics of the locomotion system and the mouth and maxillary system have led to the development of in-depth studies on the interrelation mechanisms between the mouth and maxillary system and the neuro-muscular system.

It is known, by now, that there are close sensorial and mechanical relationships between the tongue, the hyoid bone, the base of the cranium, the lower jaw, the scapular cingulum and the breastbone, the cervical-thoracic-lumbar-sacral rachis, the pelvic cingulum, the hip, knee, ankle and foot joints.

These structures constitute the so-called postural chain and their mutual connection causes the imbalance of one of these districts to be able to be transmitted to the contiguous ones and hence to the whole chain.

In particular, in the field of sports, when the lower jaw is forced to remain displaced to avoid unacceptable and dangerous contacts between the teeth of the upper arch and those of the lower arch, a muscular accommodation descending along the whole postural chain is immediately generated and is extremely tiresome. Moreover, in the long run, there is a bottom to top return of this phenomenon, which imposes an additional accommodation effort to the already compromise masticatory muscles.

It is thus known that posture control, which also entails the correct positioning of the lower jaw, assures the stabilization of the body parts that exercise a supporting action when the other parts are in motion.

In sports, the bite-positioner is the means whereby the athlete obtains an optimal dental bearing, the freedom to seek the best positioning of the lower jaw, the condition of maximum comfort for temporal-mandibular joints and, above all, the better stabilization of the head and the maximum postural balance, essential conditions to improve the motion.

### BACKGROUND ART

Known bite-positioners for sports activities essentially comprise a U-shaped support body, to copy the shape of the user's upper and lower arch, and made of soft material.

Prior art bite-positioners have a thickness, i.e. the distance between the surfaces in contact with the teeth, that is constant along their development or in any case has substantially symmetrical profile relative to a plane coinciding with the plane of symmetry of the user's mouth.

Prior art bite-positioners, studied specifically for sporting activities, are not specifically designed in order to optimize the complex sets of motions a sportsman goes through during his/her activity.

In this sporting activity, correct posture is extremely important both in balancing before the shot, and during the movement itself.

For instance, with reference to the sport of golf, in the movement that precedes the impact with the ball, the golfer exercises a considerable activity of the muscles of the scapular-humeral cingulum associated to a rotation of the shoulders to the right (left for left-handers) but with the need to maintain a perfect posture of the head, which remains motionless, both in long distance shots and in short distance shots. Moreover, in short distance shots, the spine and the head undergo a slight inclination to the right (left for left-handers).

The mouth and maxillary system undergoes a slight deviation towards the part where the rotation takes place and the sports person tends to contract more the facial muscles of that side, regardless of the presence of absence of occlusal disturbances. This situation is due to the close interdependence between the muscles of the mouth and maxillary system and those of the scapular-humeral cingulum and, consequently, of all the components of the postural chain.

The different muscular tension can therefore create imbalances on the postural system with consequences both on the complex set of motions of the arms and on the stability of the feet on the ground.

The same happens for golf players or for athletes that play some track and field events, such as shot putting, javelin throwing, hammer throwing, discus throwing and for other sports.

Prior art bite-positioners, which are substantially symmetrical, are not able to solve the drawbacks described above.

### DISCLOSURE OF THE INVENTION

An object of the present invention is to overcome the problems noted in the prior art, proposing a bite-positioner that is capable of overcoming the aforementioned drawbacks.

In particular, an object of the present invention is to obtain a bite-positioner that is able to eliminate the destabilization of the muscles of the player or athlete's mouth and jaw muscle system during the typical motions performed when playing the relating sport.

A further object of the present invention is to propose a bite-positioner that assures an improvement of the player or athlete's overall motion and which therefore enables him/her to express with continuity the maximum muscle power with maximum precision in each phase of the motion.

These objects and others beside, which shall become more readily apparent in the description that follows, are substantially achieved by a bite-positioner comprising the characteristics expressed in one or more of the appended claims.

### DESCRIPTION OF THE DRAWINGS

Further features and advantages shall become more readily apparent from the detailed description of a preferred, but not exclusive, embodiment of a bite-positioner in accordance with the present invention. Said description shall be provided hereafter with reference to the accompanying figures, provided purely by way of non limiting indication, in which:

FIG. 1 shows a perspective view of a bite-positioner in accordance with the present invention;

FIG. 2 shows a top view of the bite-positioner of FIG. 1;

FIG. 3 shows a bottom view of the bite-positioner of FIG. 1; and

FIG. 4 shows a section of the bite-positioner of FIG. 1 according to line IV-IV of FIG. 2.

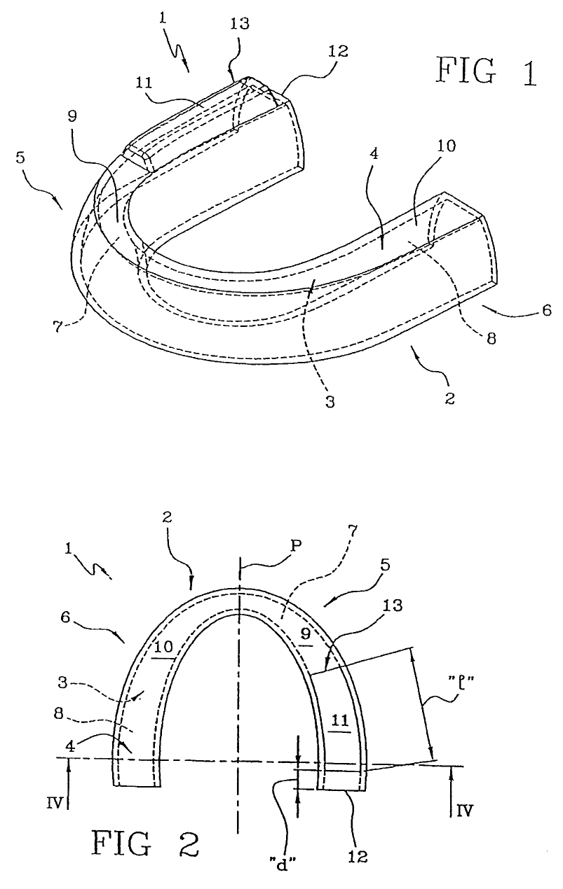

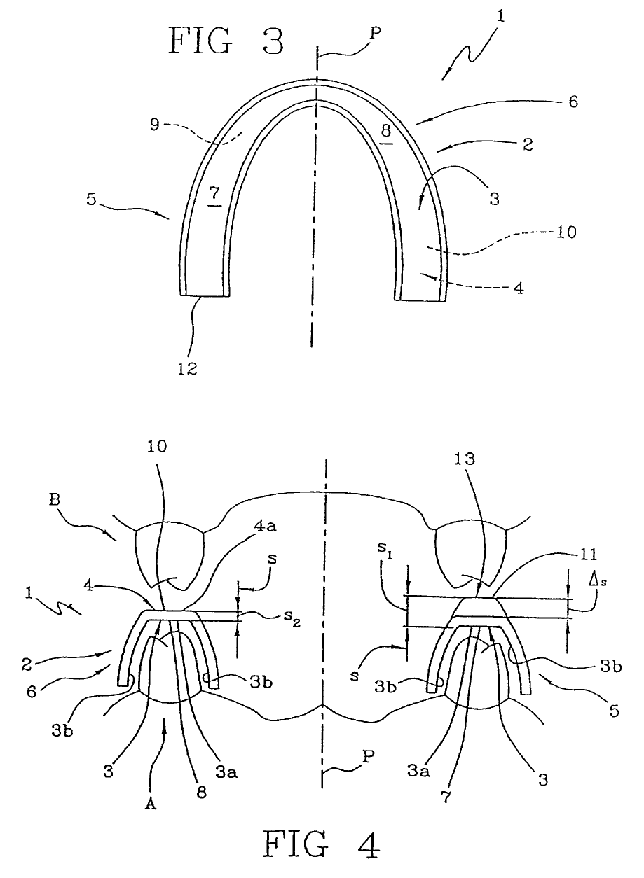

### DESCRIPTION OF THE ILLUSTRATIVE EMBODIMENT

A bite-positioner in accordance with the present invention is designated in the accompanying drawings with the reference number 1.

The bite-positioner 1 comprises a support body 2 shaped substantially as U to reproduce the shape of a user's lower and upper arches. The support body 2 has a first active surface 3 and a second active surface 4 able to be engaged respectively to the lower arch “A” and to the upper arch “B” of the user's mouth.

The support body 2, positioned in the mouth, is placed between the two arches “A” and “B” to provide the correct support to the teeth tightly shut against it.

More in detail, the support body 2 is defined by two half-parts 5, 6 which form a single body and which develop in mutually opposite directions from a median plane P coinciding with the plane of symmetry of the user's mouth. Each half-part 5, 6 of the support body can be associated to a semi-arch.

The first active surface 3 is orthogonal to the median plane P, it is substantially U-shaped and it is defined by two half surfaces 7, 8, each associated with a respective half-part 5; 6 of the support body 2. The median plane P divides the first surface 3 into the two half surfaces 7, 8, each of which comes in contact with one of the lower semi-arches.

Similarly, the second active surface 4 is orthogonal to the median plane P, it is substantially U-shaped and it is defined by two half surfaces 9, 10, each associated with a respective half-part 5; 6 of the support body 2. The median plane P divides the second surface 3 into the two half surfaces 9, 10, each of which comes in contact with one of the upper semi-arches.

The active surfaces 3, 4 develop substantially parallel to each other and are mutually separated by a determined distance which defines the useful thickness “s” of the bite-positioner 1, i.e. the thickness whereby are distanced the teeth of the two arches “A”, “B” (FIG. 4).

In the preferred embodiment, shown in the accompanying figures, the bite-positioner is of the lower type, i.e. it is made to engage to the lower arch “A” and comes in contact with the upper arch “B” only when the teeth are shut. Its second active surface 4 therefore represents the lower occlusal plane.

As is clearly shown in FIG. 4, which represents a cross section relative to the active surfaces 3, 4, the first active surface 3 has a concave shape that is substantially complementary to the user's lower arch “A”, to be anchored thereto, whilst the second active surface 4 is substantially hollow.

More specifically, the second active surface 4 is constituted by a simple segment 4a, substantially planar or anyway shaped in such a way as to assure the adhesion of the teeth, whereon bear the teeth of the upper arch “B”, whilst the first active surface 4, in addition to a substantially planer segment 3a for the bearing of the crown of the teeth of the lower arch “A”, has two lateral walls 3b which extend on the sides of the teeth themselves.

It should be noted that the useful thickness “s” represents the distance between the substantially planar segments 3a, 4a of the active surfaces 3, 4 with which the crowns of the teeth of the two arches “A”, “B” come in contact.

Advantageously, as shown in the accompanying drawings, the useful thickness “s₁” of at least one bearing portion 11 of one of the two half-parts 5, 6 is greater than the useful thickness “s₂” of the other of the two half-parts 5, 6. The bite-positioner 1 has a difference in thickness “Δs” between the two half-parts 5, 6.

In the illustrated embodiment, the bearing portion 11 with the greater useful thickness “s₁” is located to the right of the median plane P, from the point of view of the user who wears the bite-positioner. This embodiment is used by the right-handed players who perform the motion to hit the ball from right to left. A wholly similar embodiment, but with the bearing portion 11 positioned to the left is provided for left-handed players.

Advantageously, the difference in thickness “Δs” is between 1 mm and 2 mm and it preferably is 1.5 mm.

More specifically, the maximum useful thickness “s₁”, which pertains to the half-part 5; 6 with the bearing portion 11, is between 3.5 mm and 4.5 mm and the minimum useful thickness “s₂”, which pertains to the half-part 6; 5 without the bearing portion 11, is between 1.5 and 2.5 mm.

In the illustrated embodiment, the difference in thickness “Δs” is constant along the development of the bearing portion 11 but, in other embodiments not illustrated herein, said difference in thickness “Δs” can be variable along the development of said bearing portion 11, the better to adapt to the mouth of the person wearing the bite-positioner.

Advantageously, the bearing portion 11 with greater thickness extends along the respective half-part 5; 6 starting from a distance “d” ranging between 1 and 5 mm relative to an end 12 of the half-part 5, 6 for a length “l” ranging between 15 and 30 mm. In any case, the bite-positioner is made in various sizes to be able to accommodate different dimensions of the lower arches “A′” and the bearing portion 11 with greater thickness is shaped to extend from the last molar to the canine tooth of the user's semi-arch.

Preferably, the support body 2 is made of thermoplastic material and the bearing portion 11, which determines with its thickness the difference in thickness Δs between the two half-parts 5, 6 is defined by a raised element 13 applied to the body 2 of thermoplastic material.

The raised element 13 can be associated to the first active surface 3, to the second active surface 4, or inserted in an appropriate seat formed in the support body 2 and interposed between the first active surface 3 and the second active surface 4.

In the illustrated embodiment, the raised element 13 is defined by a plate made of yielding and elastic material, preferably silicone, joined to the second active surface 4.

Alternatively, according to an embodiment not illustrated herein, the bite-positioner is obtained in a single piece with a single material, for instance silicone, and shaped to have the advantageous difference in thickness, described above.

The invention achieves important advantages.

First of all, the bite-positioner according to the present invention allows to eliminate the destabilization of the muscles of the athlete's mouth and jaw system during the asymmetrical set of motions that is typical of some sports. The asymmetry of the subject bite-positioner allows to optimize posture, to obtain the correct balancing during the athletic stress. For the golf player, for instance, the correct balancing is obtained both before the shot, and during the motion.

Moreover, use of an elastic and resilient material, like silicone, assures the necessary comfort and makes the bite-positioner very well tolerated by the person who wears it.

Furthermore, the bite-positioner lowers the stress of muscles in tension during the athletic stress. For instance, the golf player that uses the bite-positioner of the invention experiences a sense of lightness in his/her arms also after playing for several hours.

Consequently, the bite-positioner according to the present invention assures the improvement of the overall set of motions and allows the athlete to express with continuity the maximum muscle power with maximum precision.

1) Bite-positioner, comprising:
a support body defined by two half-parts developing in opposite directions from a median plane;
the support body having a first active surface able to be engaged to the lower arch of a user's mouth and a second active surface able to be engaged to the upper arch of the user's mouth; the first active surface being orthogonal to said median plane, substantially U-shaped and defined by two half-surfaces, each associated to a respective half-part of the support body; the second active surface being orthogonal to said median plane, substantially U-shaped and defined by two half-surfaces, each associated to a respective half-part of the support body; said active surfaces developing substantially parallel to each other and mutually separated by a determined distance defining a useful thickness of said bite-positioner;
wherein the useful thickness of at least a bearing portion of one of the half-parts is greater than the useful thickness of the other of the two half-parts, to define a difference in thickness between said half-parts.

2) Bite-positioner according to claim 1, wherein the difference in thickness between the two half-parts is between 1 mm and 2 mm and preferably of 1.5 mm.

3) Bite-positioner according to claim 1, wherein it has a minimum useful thickness of between 1.5 mm and 2.5 mm.

4) Bite-positioner according to claim 1, wherein it has maximum useful thickness of between 3.5 mm and 4.5 mm.

5) Bite-positioner according to claim 1, wherein the difference in thickness is constant along the development of said bearing portion.

6) Bite-positioner according to claim 1, wherein the difference in thickness is variable along the development of said bearing portion.

7) Bite-positioner according to claim 1, wherein one of said two half-parts has a raised element which defines the bearing portion and determines said difference in thickness.

8) Bite-positioner according to claim 7, wherein said raised element is associated to the first active surface.

9) Bite-positioner according to claim 7, wherein said raised element is associated to the second active surface.

10) Bite-positioner according to claim 7, wherein said raised element is interposed between the first active surface and the second active surface.

11) Bite-positioner according to claim 7, wherein raised element is made of an elastic material.

12) Bite-positioner according to claim 7, wherein said raised element is made of yielding material.

13) Bite-positioner according to claim 7, wherein said raised element is made of silicone.

14) Bite-positioner according to claim 1, wherein said at least a bearing surface with greater thickness extends along the respective half-part for a length of between 15 mm and 30 mm.

15) Bite-positioner according to claim 1, wherein said at least a bearing portion with greater thickness extends starting from a distance of between 1 mm and 5 mm relative an end of said half-part.

16) Bite-positioner according to claim 1, wherein said at least a bearing portion with greater portion extends from the last molar to the canine tooth of the user's semi-arch.

17) Bite-positioner according to claim 1, wherein the support body is made of thermoplastic material.

18) Bite-positioner according to claim 1, wherein it is a lower bite-positioner.

19) Bite-positioner according to claim 1 wherein, in a cross section to the active surfaces, the first active surface has a concave shape, substantially complementary to the user's lower arch, to be anchored to said lower arch.

20) Bite-positioner according to claim 1, wherein it is defined by a single piece and by a single material and it is shaped to have said difference in thickness.
